(19) United States
(12) Patent Application Publication
(10) Pub. No.: US 2003/0185865 A1
(43) Pub. Date: Oct. 2, 2003

(54) Cosmetic or dermatological preparations for avoiding skin damage by peroxide

(76) Inventors: Axel Jentzsch, Mannheim (DE); Heinz Friedrich Sutoris, Worms (DE); Gerhard Wagenblast, Worms (DE); Sylke Haremza, Neckargemund (DE)

Correspondence Address: KEIL & WEINKAUF, 1350 CONNECTICUT AVENUE, N.W., WASHINGTON, DC, 20036, US

(21) Appl. No.: 10/333,613

(22) PCT Filed: Jul. 19, 2001
(86) PCT No.: PCT/EP01/08358

(30) Foreign Application Priority Data
Jul. 26, 2000 (DE) 100 36 655.4

(51) Int. Cl. A61K 7/00

(52) U.S. Cl. 424/401

(57) ABSTRACT

Cosmetic or dermatological preparations for avoiding or reducing skin damage by peroxides or hydroperoxides formed as a result of endogenous or exogenous factors, with a content of

a) at least one antioxidant effective as O- or C-free radical scavenger and

b) at least one organic or inorganic skin-compatible compound which reduces peroxides or hydroperoxides to the corresponding alcohols without the formation of active free-radical subsequent stages, where this compound is chosen such that, at body temperature, it reacts significantly more rapidly than sulfur-containing compounds intrinsic to the skin.

The invention relates to the use of a combination of antioxidants and peroxide decomposers which react, by reduction without the formation of free-radical subsequent stages with the peroxides, with peroxides or hydroperoxides more rapidly than sulfur-containing compounds intrinsic to the skin, and to cosmetic and dermatological preparations which comprise this combination.

The human skin is subject to certain aging processes, some of which are to be attributed to intrinsic to the skin processes (chronoaging) and some of which are to be attributed to exogenous factors (environmental, e.g. photoaging). In addition, temporary and also permanent changes in the appearance of the skin can arise, such as acne, greasy or dry skin, keratoses, rosaceae, light-sensitive, inflammatory, erythematous, allergic or autoimmune reactions, such as dermatoses, photodermatoses and others, the exact causes of which and factors which influence them often only being partly understood.

Exogenous factors include, in particular, sunlight or artificial radiation sources with a comparable spectrum, and compounds which can arise as a result of the radiation, such as undefined reactive photoproducts, which may also be free-radical or ionic. However, these factors also include harmful or reactive compounds such as ozone, free-radicals, for example the hydroxyl radical, singlet oxygen and other reactive oxygen or nitrogen compounds, cigarette smoke, natural and synthetic toxins, and others which interfere with the natural physiology or morphology of the skin. The effect of these factors may result inter alia in direct damage to the DNA of the skin cells, and to the collagen, elastin or glycosaminoglycan molecules of the extracellular matrix which are responsible for the firmness of the skin. Moreover, signal transduction chains may be affected, resulting in the activation of harmful factors, e.g. matrix-degrading enzymes. Important representatives of these enzymes are the matrix metalloproteinases (MMPs, e.g. collagenases, gelatinases, stromelysines), the activity of which is additionally regulated by TIMPs (tissue inhibitor of matrix metalloproteinases).

In addition, the harmful effects lead to damage of the cells of the skin itself. As a consequence thereof, the regeneration ability of the skin, for example, is reduced.

A further consequence may be inflammatory reactions, and, inter alia, immunoregulatory compounds, such as interleukins, prostaglandins and histamines, are released. As a result, immunocompetent cells are attracted, inter alia, and the inflammatory reaction is intensified.

The consequences of aging are thinning of the skin, weaker meshing of epidermis and dermis, reduction in cell number and in supplying blood vessels. The aging processes lead to the formation of fine lines and wrinkles, the skin becomes leathery, yellowish and starts to sag, and pigment disorders arise.

Compounds which have an antioxidative effect are often used in dermatological or cosmetic preparations for protecting against decay. Moreover, they can, however, also be used in order to reduce harmful or undesired oxidative processes which occur in human or animal skin. It is known that such processes play a significant role in skin aging. The skin is exposed to permanent oxidative stress by the formation of peroxides and hydroperoxides, some of which originate from the external environment of the skin, but some of which are also formed endogenously. In order to counteract this stress, the skin has a large number of its own protective mechanisms. These protective mechanisms, however, are insufficient to prevent oxidative processes in the skin completely. By contrast, it is generally assumed that these very oxidative processes make a significant contribution to skin aging, but also to general or pathological changes in the skin.

In particular, the importance of lipid peroxidation for aging is generally recognized. The toxic effect of lipid hydroperoxides and their decomposition products has also been described by W. A. Prior (ACS Sysup. Ser. (1985), 277, 77-96), inter alia. For the decomposition of peroxides, hydroperoxides or hydrogen peroxide, various systems have also been described in connection with cosmetics, for example the use of metallophosphyrines (JP 3273082), phytic acid zinc salts (JP 08104635), catalase (JP 08175035) and other enzymes (JP 67165553). In addition, JP 06345797 discloses the use of cysteine-containing dipeptides for the bleaching of skin, for the prevention of lipid peroxidation and for the decomposition of lipid peroxides.

To aid the endogenous protective mechanisms, constituents with an antioxidative effect, i.e. effective as O- or C-free-radical scavengers, are therefore added to cosmetic and dermatological preparations (e.g. DE 19739349). However, the effect actually achieved has hitherto fallen short of that hoped for. In particular, an increase in the added amount of antioxidant does not usually achieve a correspondingly higher antioxidative effect.

It is an object of the present invention to propose a system of active ingredients for use in cosmetic or dermatological preparations with which the antioxidative effect can be considerably increased.

In general, the mechanism of the formation of peroxide or hydroperoxide conforms to the following scheme

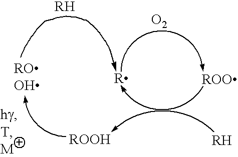

While the customary antioxidants are essentially O- or C-free-radical scavengers, it is an object of the invention to prevent skin damage more efficiently by further measures by intervention in the mechanism of this scheme additionally at another site. For this, an ionic and reducing attack according to the following scheme was considered.

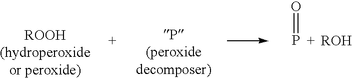

It has now been found, surprisingly, that the use of a combination of an antioxidant as free-radical scavenger and a peroxide decomposer with a reducing action has an excellent synergistic effect. In this connection, the peroxide decomposer must be chosen such that it is significantly more reactive in vitro than correspondingly effective sulfur-containing compounds intrinsic to the skin, such as cystine or cysteine.

In particular, we have found that the object is achieved with cosmetic or dermatological preparations for avoiding or reducing skin damage by peroxides or hydroperoxides formed by endogenous or exogenous factors which comprise an effective content of

a) at least one antioxidant effective as O- or C-free-radical scavenger and

b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin.

The cosmetic or dermatological preparations usually comprise, based on the finished preparations, 0.001 to 30% by weight, preferably 0.01 to 10% by weight and in particular 1 to 5% by weight, of antioxidant (a) and 0.001 to 30% by weight, preferably 0.01 to 10% by weight and in particular 1 to 5% by weight, of at least one peroxide or hydroperoxide decomposer (b).

The peroxide or hydroperoxide decomposers (b) may belong to very diverse classes of chemical compounds. In this connection, it goes without saying that only skin-compatible representatives or skin-compatible concentrations of these classes of compound are suitable. In addition, they must have a significantly greater decomposing (reducing) action than compounds intrinsic to the skin, such as cystine or cysteine. Whether certain compounds are suitable for the use according to the invention can be seen in vitro, for example, from the fact that, at room temperature, dissolved in a molar concentration of 0.05 m/l in a polar or nonpolar solvent, they reduce the peroxide or hydroperoxide concentration by at least 20%, preferably 50% and in particular 90%, within 3 minutes.

Specifically, suitable classes of compound are sulfur-containing compounds in which the sulfur is present in an oxidation state of less than +6, phosphorus-containing compounds in which the phosphorus is present in an oxidation state of less than +5, and aromatic amines. The sulfur- or phosphorus-containing compounds may be organic or inorganic, preference being given to organic compounds.

Suitable sulfur-containing classes of compound are mercaptans, dialkyl, diaryl or arylalkyl sulfides, dialkyl disulfides, dialkyl sulfoxides, sulfinic acids, and esters and amides thereof, sulfenic acid esters or amides, thioesters, thioamides, thioureas, thiocarbonyl compounds and thioacetals and -ketals, including those in cyclic form. Examples which may be mentioned are sodium sulfite, sodium bisulfite, sodium thiosulfate and particularly preferably 5-thiapalmitic acid, thiobenzamide and 2-mercaptoimidazole.

Suitable phosphorus-containing compounds are phosphines or oxygen-containing phosphorus compounds, e.g. orthophosphorous acid or an ester of orthophosphorous acid. Esters of orthophosphorous acid are also referred to as phosphites. The orthophosphorous acid may also be in the form of a salt (in most cases in the form of an alkali metal or ammonium salt). Preferred bonding partners of phosphorus are the elements C, S, O, N and/or H.

Also suitable are, in particular, the phosphonites (esters of phosphonous acid) known as stabilizers.

Particularly suitable phosphites (i.e. esters of orthophosphorous acid) and phosphonites (esters of phosphonous acid) include, for example, triphenyl phosphite, diphenylalkyl phosphite, phenyldialkyl phosphite, tris(nonylphenyl) phosphite, trilauryl phosphite, tris(O-tocopheryl) phosphite, trioctadecyl phosphite, distearylpentaerythritol diphosphite, tris(2,4-di-tert-butylphenyl) phosphite, diisodecylpentaerythritol diphosphite, bis(2,4-di-tert-butylphenyl)pentaerythritol diphosphite, bis(2,6-di-tert-butyl-4-methylphenyl)pentaerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl)-4,4′-biphenylene diphosphite, tetrakis(2,4-di-tert-butylphenyl)-4,4′-biphenylene diphosphonite, 6-isooctyloxy-2,4,8,10-tetra-tert-butyl-12H-dibenzo[d,g]-1,3,2-dioxaphosphocine, 6-fluoro-2,4,8,10-tetra-tert-butyl-12-methyldibenzo[d,g]-1,3,2-dioxaphosphocine, bis(2,4-di-tert-butyl-6-methylphenyl)methyl phosphite, bis(2,4-di-tert-butyl-6-methylphenyl)ethyl phosphite and triphenylphosphine.

Advantageously, use is made here of esters of orthophosphorous acid (phosphites) of the formula (I) or esters of phosphonous acid (phosphonites) of the formula (II)

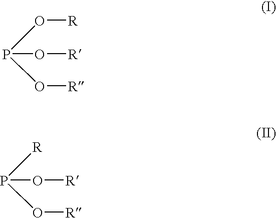

where R, R′, R″ may be identical or different and are organic radicals, in particular C₁-C₂₀-alkyl, hydroxylalkyl having 2 to 4 carbon atoms, haloalkyl, in particular chloroalkyl having 2 to 4 carbon atoms, aryl, in particular phenyl or aryl substituted by C₁-C₈-alkyl (in particular phenyl substituted by C₁-C₄-alkyl). It is also possible for two of the three organic radicals R, R′and R″, together with the phosphorus and the two oxygen atoms, to form a heterocycle (for example 5- or 6-atomed).

Names which may be given are trimethyl, triethyl, tributyl, trihexyl, trioctyl, triphenyl, tri-p-cresyl, trixylyl, tritolyl and tri-β-chloroethyl phosphite. Also suitable, however, are dimethyl, diethyl, dibutyl, dioctyl, diphenyl, ditolyl and dixylyl phosphites. Particularly suitable products are those known under the trade names Irgafos® 68 (Ciba AG), Irgafos® P-EPQ (Ciba AG) or Ultranox® 626 (GE-Speciality Chemicals GmbH).

Suitable amines are primarily secondary amines having at least one aryl radical e.g. of the formula III

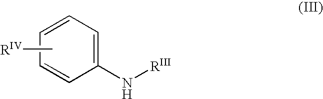

in which R^{III} is a low molecular weight alkyl radical or an aryl radical and R^{IV} is a low molecular weight alkyl or alkoxy. Specifically, compounds of the formula III may be diphenylamine derivatives, or else heterocyclic compounds in which R^{III} forms a ring with the phenyl radical.

Examples which may be mentioned are phenothiazine of the formula (IIIa), and 2-methoxyphenothiazine.

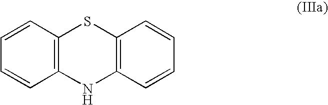

The abovementioned peroxide decomposers may be hydrophilic and/or lipophilic and, correspondingly, dissolve in the oil phase, or in the water phase, respectively.

Particular preference is given to organic sulfur- and/or phosphorus-containing compounds.

Specifically, mention may be made by way of particular preference of the following compounds:

2,2,4-trimethyl-6-ethoxy-1,2-dihydroquinoline (ethoxyquine), the compound of the formula IV

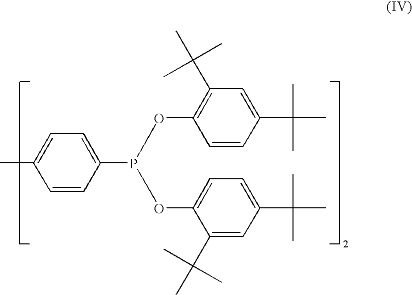

sodium thiosulfate and also 5-thiapalmitic acid, thiobenzamide and 2-mercaptoimidazole.

The choice from the abovementioned classes of compound is made on the basis of the conditions of skin compatibility or skin-compatible concentration and the effectiveness of the peroxide or hydroperoxide decomposition. For this purpose, the compound under consideration is dissolved in a polar solvent (e.g. acetic acid) or a nonpolar solvent (e.g. toluene) in a molar concentration of 0.05 m/l, and the decomposition rate of a peroxide or hydroperoxide over the course of 3 minutes is measured. In this connection, the concentration of the peroxide or hydroperoxide should be decreased by at least 20%, preferably 50% and in particular 90%.

The antioxidants (a) are usually compounds known per se. The antioxidants are advantageously chosen from the group of carotenoids, carotenes (e.g. α-carotene, β-carotene, lycopene) and derivatives thereof, chlorogenic acid and derivatives thereof, lipoic acid and derivatives thereof (e.g. dihydrolipoic acid), and also (metal) chelating agents, EDTA, EGTA and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), butylhydroxytoluene, butylhydroxyanisole, and further antioxidants customarily used in cosmetic preparations.

The amount of the abovementioned antioxidants (a) in the finished preparations is, for example, 0.001 to 30% by weight, preferably 0.01 to 10% by weight and in particular 1 to 5% by weight.

The cosmetic and dermatological preparations according to the invention offer effective protection against

oxidative processes,

processes caused by radiation or reactive compounds.

With regard to their other constituents, the novel cosmetic and dermatological formulations can have the customary composition and be used for the treatment, care and cleansing of the skin in cosmetics. The composition depends here on the effectiveness of the inhibitor, the penetration properties of the active substance through the Stratum corneum and its ability to form a depot in the skin.

Surprisingly, application according to the invention of the active ingredient combination permits a cosmetically effective treatment, but also prevention of

prematurely aged skin (e.g. wrinkles, age spots, teleangiectases, pigment disorders) and/or prematurely aged skin appendages

radiation-induced skin damage or radiation-induced negative changes in the skin and/or the skin appendages

environmentally induced (ozone, free-radicals, singlet oxygen, reactive oxygen or nitrogen compounds, cigarette smoke, toxins) skin damage or environmentally induced negative changes in the skin and/or the skin appendages

light-sensitive, inflammatory, erythematous, allergic or autoimmune reactive changes in the skin and/or the skin appendages (in particular acne, greasy or dry skin, keratoses, rosaceae, dermatoses, atopic eczema, seborrhoic eczema, photodermatoses, polymorphous light dermatosis)

deficient, sensitive or hypoactive states of the skin and/or the skin appendages

itching and

dry skin states and horny layer barrier disorders.

For use, the cosmetic and dermatological preparations according to the invention are applied to the skin (and/or the hair) in a sufficient amount in the manner customary for cosmetics.

For example, the active ingredients according to the invention are used in cosmetic compositions for the cleansing of the skin, such as bar soaps, toilet soaps, curd soaps, transparent soaps, luxury soaps, deodorizing soaps, cream soaps, baby soaps, skin protection soaps, abrasive soaps, syndets, liquid soaps, pasty soaps, soft soaps, washing pastes, liquid washing, showering and bath preparations, e.g. washing lotions, shower preparations, shower gels, foam baths, cream foam baths, oil baths, bath extracts, scrub preparations, in-situ products, shaving foams, shaving lotions, shaving creams.

In addition, they are suitable for skin cosmetic preparations, such as W/O or O/W skin and body creams, day and night creams, light protection compositions, aftersun products, hand care products, face creams, multiple emulsions, gelees, microemulsions, liposome preparations, niosome preparations, antiwrinkle creams, face oils, lipogels, sport gels, moisturizing creams, bleaching creams, vitamin creams, skin lotions, care lotions, ampoules, aftershave lotions, preshaves, humectant lotions, tanning lotions, cellulite creams, depigmentation compositions, massage preparations, body powders, face tonics, deodorants, antiperspirants, nose strips, antiacne compositions, repellents and others.

In addition, the active ingredients according to the invention can be used in cosmetic compositions for hair care, such as hair cures, hair lotions, hair rinses, hair emulsions, split-end fluids, neutralizing agents for permanent waves, hot-oil treatment preparations, conditioners, setting lotions, shampoos, hair tints and colorants, hairsprays, blow-waving lotions, blow-waving setting compositions, shine sprays, hair brillantines, hair-styling products, hair tonics, alopecia care compositions and others.

The cosmetic or dermatological preparations can, depending on the field of use, be in the form of a spray (pump spray or aerosol), foam, gel, gel spray, lotion, cream, mousse, ointment, suspensions or powders.

It is also advantageous to administer the active ingredients in encapsulated form, e.g. as cellulose encapsulation, in gelatin, with polyamides, in niosomes, wax matrices, with cyclodextrins or liposomally encapsulated.

The preparations according to the invention generally comprise further auxiliaries as are customarily used in such preparations, e.g. preservatives, bactericides, perfumes, antifoams, dyes, pigments, thickeners, surface-active substances, emulsifiers, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents or silicone derivatives.

In addition to said additives, the preparations according to the invention can comprise further compounds which have an antioxidative, free-radical scavenger, skin moisturizing or moisture-retaining, antierythematous, antiinflammatory or antiallergic action, in order to supplement or enhance their action. In particular, these compounds can be chosen from the group of vitamins, plant extracts, alpha- and-beta-hydroxy acids, ceramides, antiinflammatory, antimicrobial or UV-filtering substances, and derivatives thereof and mixtures thereof.

Advantageously, preparations according to the invention can also comprise substances which absorb UV radiation in the UV-B and/or UV-A region.

The lipid phase is advantageously chosen from the group of substances of mineral oils, mineral waxes, branched and/or unbranched hydrocarbons and hydrocarbon waxes, triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures; oils, fats or waxes, esters of saturated and/or unsaturated, branched and/or unbranched C₃-C₃₀-alkanecarboxylic acids and saturated and/or unsaturated, branched and/or unbranched C₃-C₃₀-alcohols, from aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil, alkyl benzoates or silicone oils, such as, for example, cyclomethicone, dimethylpolysiloxane, diethylpolysiloxane, octamethylcyclotetrasiloxane and mixtures thereof or dialkyl ethers.

The aqueous phase of the preparations according to the invention optionally advantageously comprises alcohols, diols or polyols of low carbon number, and ethers thereof, preferably ethanol, isopropanol, propylene glycol, glycerol, ethylene glycol monoethyl ether.

Suitable emulsifiers are preferably known W/O and also O/W emulsifiers, such as polyglycerol esters, sorbitan esters or partially esterified glycerides.

Suitable solubility promoters are, in particular, ethoxylated sorbitan esters, ethoxylated lanolin alcohols and ethoxylated castor oil.

Customary native and synthetic thickeners or gel formers in formulations are crosslinked polyacrylic acids and derivatives thereof, polysaccharides, such as xanthan gum or alginates, carboxymethylcellulose or hydroxycarboxymethylcellulose, hydrocolloids such as gum arabic or montmorillonite minerals, such as bentonites or fatty alcohols, polyvinyl alcohol and polyvinylpyrrolidone.

Suitable propellants for aerosols according to the invention are the customary propellants, for example propane, butane, pentane and others.

### EXAMPLE 1

### Measurement of the Peroxide Decomposition

The compounds to be used according to the invention listed in Table 1 and 2 were investigated with regard to their peroxide-decomposing action compared with cystine and cysteine in accordance with the experimental arrangement given below.

### Description of the Experiment

The following solutions were prepared:

1. 0.05 molar solution of tert-butyl hydroperoxide in CD₃COOD

2. 0.055 molar solution of the potential hydroperoxide decomposer in CD₃COOD

From these, the measurement solutions were prepared by mixing 350 μl of solution 1 and 350 μl of the respective solution 2; the measurement solution was then introduced into an NMR tube and transferred to the NMR instrument. Preparation of these solutions and taking of the measurements was always carried out at 23° C. The time until measurements were taken was about 3 minutes. All of the measurements were carried out using an INOVA 500 500 MHz NMR spectrometer from Varian. For each measurement solution, a ¹H-NMR spectrum and a 2D-HSQC (¹H/¹³C) spectrum were recorded. Tert-butylhydroperoxide and tert-butanol each had CH₃ proton signals which were very close together; assignment of the signals to tBuOOH or tBuOH was made by reference to the 2D-HSQC spectra. The relative proportions of the two components were ascertained by integration of the signal of the corresponding components in the ¹H spectrum or of the crosspeaks in the HSQC spectrum (Lit: W. Wilker et al. Magn. Reson. Chem. 31, 287-292 (1993)).

A further test series was carried out analogously to the above experimental series in deuterated toluene (=N) instead of CD₃COOD (=S).

In each case, 350 μl of 0174 (A80) and 350 μl of the other samples were mixed. The solvent used was toluene-d8 (=N) or CD₃COOD (=S).

### Example

Peroxide or hydroperoxide decomposer (b):

Solvent: CD₃COOD (=S), toluene-D₈ (=N)

### Comparison Compounds (Not in Accordance with the Invention)

| | | | t-BuOOH reduction |
|---|---|---|---|
| No. | Substance | Solvent | (%) at 22° C. in 3 min |
| 1 | L-Cystine | S | 0% |
| 2 | L-Cysteine | S | 0% |
| 3 | S-Benzyl-L-cysteine | S | 0% |
| 4 | L-Methionine | S | 4% |
| 5 | D(+)-Biotin | S | 0% |
| 6 | T-α-Lipoic acid | S | 3% |

### P Compounds According to the Invention

| | | | t-BuOOH reduction |
|---|---|---|---|
| No. | Substance | Solvent | (%) at 22° C. in 3 min |
| 7 | Triphenylphosphine | N | 100% |
| 8 | Triethyl phosphite | N | 22% |
| 9 | Trisnonylphenyl phosphite | S | 29% |
| 10 | Irgafos PEP-Q *) | N | 80% |
| 11 | Tris(O-tocopheryl) phosphite | N | 22% |

### S-Compounds According to the Invention

| | | | t-BuOOH reduction |
|---|---|---|---|
| No. | Substance | Solvent | (%) at 22° C. in 3 min |
| 12 | 5-Thiapalmitic acid | S | 26% |
| 13 | Thiobenzamide | S | 59% |
| 14 | 2-Mercaptoimidazole | S | 21% |
| 15 | Sodium sulfite | S | 40% |
| 16 | Sodium bisulfite | S | 33% |

### Aromatic Amines According to the Invention

| | | | t-BuOOH reduction |
|---|---|---|---|
| No. | Substance | Solvent | (%) at 22° C. in 3 min |
| 17 | Phenothiazine | S | 22% |
| 18 | 2-Methoxyphenothiazine | S | 25% |
| 19 | Ethoxyquine | N | 67% |

*) Irgafos-P-EPQ = Compound of the formula IV

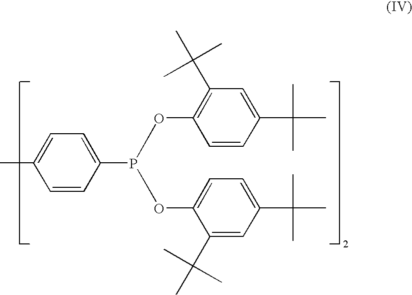

### Examples of Cosmetic Preparations

| | | Example |
|---|---|---|
| Formulation type | Area of application | No. |
| O/W emulsion | Soft skin lotion | 1-13 |
| W/O emulsion | Hand protection cream | 14-26 |
| | Sun care lotion | 27-39 |
| Multiple emulsion | W/O/W emulsion | 40-52 |
| Microemulsion | Microemulsion | 53-65 |
| Hydrophilic gel | Liposome gel | 66-78 |
| Lipophilic gel | Blunted oil gel | 79-91 |
| | Oil gel | 92-104 |
| Stick formulation | Sun care-lip protection | 105-117 |
| | stick | |
| Aqueous cosmetics | Cooling body splash | 118-130 |
| Decorative cosmetics | Make-up | 131-143 |
| | Liquid make-up | 144-156 |
| Oils | Sun care oil | 157-169 |
| Body cleansing composition | Facial scrub cleanser | 170-182 |
| Hair aftertreatment rinse- | Conditioner | 183-195 |
| off | | |
| Hair aftertreatment leave- | Hair wax | 196-208 |
| in | Antidandruff hair tonic | 209-221 |
| Aerosol | Foot deospray | 222-234 |
| | Hair spray | 235-247 |

Formulations 1-13—Soft skin fluid

| | % w/w |
|---|---|
| Ceteareth-6 and stearyl alcohol | 2.50 |
| Ceteareth-25 | 2.50 |
| Hydrogenated cocoglycerides | 1.50 |
| PEG-40 dodecyl glycol copolymer | 3.00 |
| Dimethicone | 3.00 |
| Phenethyl dimethicone | 2.00 |
| Cyclomethicone | 1.00 |
| Cetearyl octanoate | 5.00 |
| Avocado oil | 1.00 |
| Sweet almond oil | 2.00 |
| Wheatgerm oil | 0.80 |
| Panthenol USP | 1.00 |
| Phytantriol | 0.20 |
| Tocopheryl acetate | 0.30 |
| Propylene glycol | 5.00 |
| Peroxide decomposer as in examples 7 to 19 | 1.00 |
| Sodium ascorbyl phosphate | 2.00 |
| Parfum | q.s. |
| Preservative | q.s. |
| Aqua | ad 100 |

Formulations 14 to 26—Hand protection cream

| | % w/w |
|---|---|
| Cetearyl alcohol | 1.00 |
| Glyceryl stearate | 1.50 |
| Stearyl alcohol | 1.50 |
| Cetyl palmitate | 2.00 |
| Tocopheryl acetate | 0.50 |
| Dimethicone | 8.00 |
| Ceteareth-6 and stearyl alcohol | 3.00 |
| Octyl methoxycinnamate | 5.00 |
| Propylene glycol | 8.00 |
| Panthenol | 1.00 |
| Evening primrose oil | 3.00 |
| PEG-7 hydrogenated castor oil | 6.00 |
| Glyceryl oleate | 1.00 |
| Phenethyl dimethicone | 3.00 |
| Beeswax | 1.50 |
| Locust bean gum | 0.80 |
| Silk powder | 0.80 |
| Borax | 0.10 |
| Preservative | q.s. |
| Parfum | q.s. |
| Peroxide decomposer as in examples 7 to 19 | 1.20 |
| Aqua | ad 100 |

Formulations 27 to 39—Sun care lotion

| | % w/w |
|---|---|
| PEG-7 hydrogenated castor oil | 6.00 |
| PEG-40 hydrogenated castor oil | 0.50 |
| Isopropyl palmitate | 7.00 |
| PEG-45/dodecyl glycol copolymer | 2.00 |
| Jojoba oil | 3.00 |
| Magnesium stearate | 0.60 |
| Octyl methoxycinnamate | 8.00 |
| C 12-15 alkyl benzoate | 5.00 |
| Titanium dioxide | 4.00 |
| Propylene glycol | 5.00 |
| EDTA | 0.20 |
| Preservative | q.s. |
| Sodium ascorbyl phosphate | 1.00 |
| Tocopheryl acetate | 0.50 |
| Peroxide decomposer as in examples 7 to 19 | 0.05 |
| Parfum | q.s. |
| Aqua | ad 100 |

Formulations 40 to 52—multiple emulsion

| | % w/w |
|---|---|
| Mineral oil | 7.50 |
| Cetearyl octanoate | 2.50 |
| Aluminum stearate | 0.25 |
| Magnesium stearate | 0.25 |
| Microcrystalline wax H | 0.50 |
| Cetearyl alcohol | 1.00 |
| Lanolin alcohol | 1.50 |
| Mineral alcohol and lanolin alcohol | 1.50 |
| PEG-7 hydrogenated castor oil | 0.75 |
| PEG-45/dodecyl glycol copolymer | 2.00 |
| Tocopheryl acetate | 3.50 |
| Ceteareth-6 and stearyl alcohol | 2.00 |
| Ceteareth-25 | 2.00 |
| Trilauret-4 phosphate | 1.00 |
| Hydroxyethylcellulose | 0.20 |
| Propylene glycol | 7.50 |
| Magnesium sulfate | 0.25 |
| Peroxide decomposer according to examples 7 to 19 | 2.00 |
| Aqua | ad 100 |

Formulations 53 to 65—Micruemulsion

| | % w/w |
|---|---|
| Ceteareth-25 | 13.00 |
| PEG-7 glyceryl cocoate | 20.00 |
| Octyldodecanol | 5.00 |
| Sodium ascorbyl phosphate | 0.50 |
| Peroxide decomposer as in examples 7 to 19 | 0.80 |
| Preservative | q.s. |
| Aqua | ad 100 |

Formulations 66 to 78—Liposome gel

| | % w/w |
|---|---|
| PEG-40 hydrogenated castor oil | 1.00 |
| Bisabolol rac. | 0.10 |
| Propylene glycol | 8.00 |
| Panthenol | 0.50 |
| Water and tocopheryl acetate and polysorbate 80 and | 3.00 |
| caprylic/capric triglyceride and lecithin | |
| Preservative | q.s. |
| Parfum | q.s. |
| Carbomer | 0.50 |
| Peroxide decomposer as in examples 7 to 19 | 0.80 |
| Triethanolamine | 0.70 |
| Aqua | ad 100 |

Formulations 79 to 91—Blunted oil gel

| | % w/w |
|---|---|
| Silica | 5.00 |
| Dimethicone | 10.00 |
| Cetearyl octanoate | 40.00 |
| Caprylic/capric triglyceride | 8.00 |
| Phenethyl dimethicone | 2.00 |
| Mineral oil | 26.00 |
| Sweet almond oil | 5.00 |
| Tocopheryl acetate | 1.00 |
| Phytantriol | 0.30 |
| Peroxide decomposer as in examples 7 to 19 | 1.50 |
| Tocopherol | 0.50 |
| Parfum | 0.70 |

FormulationS 92 to 104—Oil gel

| | % w/w |
|---|---|
| Silica | 5.00 |
| Dimethicone | 10.00 |
| Cetearyl octanoate | 30.00 |
| Isopropyl myristate | 5.00 |
| Caprylic/capric triglyceride | 10.00 |
| Phenethyl dimethicone | 5.00 |
| Mineral oil | 25.70 |
| Jojoba oil | 5.00 |
| Tocopheryl acetate | 1.00 |
| Phytantriol | 0.30 |
| Peroxide decomposer as in examples 7 to 19 | 1.50 |
| Tocopherol | 0.50 |
| Parfum | 1.00 |

Formulations 105 to 117—Sun care lip protection stick

| | % w/w |
|---|---|
| Beeswax | 12.00 |
| Hydrogenated cocoglycerides | 5.00 |
| Ricinus oil | 40.00 |
| Isopropyl palmitate | 10.00 |
| Mineral oil | 7.50 |
| Candellila wax | 8.00 |
| Phenethyl dimethicone | 5.00 |
| Tocopheryl acetate | 1.00 |
| Peroxide decomposer as in examples 7 to 19 | 1.50 |
| Petrolatum | 5.00 |
| Benzophenone-3 | 5.00 |

Formulations 118 to 130—Cooling body splash

| | % w/w |
|---|---|
| PEG-40 hydrogenated castor oil | 2.00 |
| Menthyl lactate | 0.20 |
| Alcohol | 5.00 |
| PEG-7 glyceryl cocoate | 2.00 |
| Witch hazel | 5.00 |
| Allantoin | 0.10 |
| Bisabolol rac. | 0.20 |
| Propylene glycol | 5.00 |
| Tocopheryl acetate | 1.00 |
| Sodium ascorbyl phosphate | 0.20 |
| Panthenol USP | 0.50 |
| Lactic acid (80%) | 0.20 |
| Peroxide decomposer as in examples 7 to 19 | 2.50 |
| Parfum | q.s. |
| Aqua | ad 100 |

Formulations 131 to 143—Make-up

| | % w/w |
|---|---|
| Ceteareth-6 and stearyl alcohol | 9.00 |
| Dimethicone | 5.00 |
| Cetearyl octanoate | 8.00 |
| Macadamia nut oil | 5.00 |
| Propylene glycol | 5.00 |
| Aqua | 53.00 |
| Sicovit White E 171 | 8.00 |
| Sicomet Brown 70 13E 2717 | 2.00 |
| Tocopheryl acetate | 0.20 |
| Peroxide decomposer as in examples 7 to 19 | 0.50 |
| Parfum | q.s. |
| Benzophenone-3 | 4.30 |

Formulations 144 to 156—Fluid make-up

| | % w/w |
|---|---|
| Ceteareth-6 and stearyl alcohol | 7.00 |
| Ceteareth-25 | 5.00 |
| Dimethicone | 5.00 |
| Cetearyl octanoate | 8.00 |
| Macadamia nut oil | 5.00 |
| Propylene glycol | 5.00 |
| Aqua | 53.00 |
| Sicovit White E 171 | 8.00 |
| Sicomet Brown 70 13E 3717 | 1.00 |
| Tocopheryl acetate | 0.20 |
| Peroxide decomposer as in examples 7 to 19 | 0.50 |
| Parfum | q.s. |
| Benzophenone-3 | 4.30 |

Formulations 157 to 169—Sun care oil

| | % w/w |
|---|---|
| Cetearyl octanoate | 38.00 |
| Caprylic/capric triglyceride | 28.20 |
| Evening primrose oil | 3.00 |
| Macadamia nut oil | 5.00 |
| Isopropyl palmitate | 5.00 |
| Dimethicone | 3.00 |
| Octyl methoxycinnamate | 8.00 |
| Octocrylene | 5.00 |
| Benzophenone-3 | 2.00 |
| Tocopheryl acetate | 2.00 |
| Phyantriol | 0.10 |
| Peroxide decomposer as in examples 7 to 19 | 0.50 |
| Tocopheryl acetate | 0.20 |
| Parfum | q.s. |

Formulations 170 to 182—Facial scrub cleanser

| | % w/w |
|---|---|
| Cocoamidopropylbetaine | 5.00 |
| Potassium cocohydrolyzed animal protein | 7.00 |
| PEG-40 hydrogenated castor oil | 2.00 |
| Polyquaternium-44 | 7.70 |
| Tocopheryl acetate | 1.00 |
| Bisabolol rac. | 0.20 |
| Panthenol | 1.00 |
| Parfum | 0.50 |
| Hydroxyethylcellulose | 2.00 |
| Peroxide decomposer as in examples 7 to 19 | 1.00 |
| Propylene glycol | 5.00 |
| Jojoba wax | 3.00 |
| Aqua | ad 100 |

Formulation 183 to 195—Conditioner

| | % w/w |
|---|---|
| Ceteareth-6 and stearyl alcohol | 2.00 |
| Ceteareth-25 | 1.00 |
| Cetearyl octanoate | 6.00 |
| Ceteareth-3 | 2.00 |
| Cetearyl alcohol | 6.00 |
| Phytantriol | 1.00 |
| Propylene glycol | 4.00 |
| Polyquaternium-11 | 5.00 |
| Tocopheryl acetate | 1.00 |
| Panthenol | 1.00 |
| Retinyl acetate | 0.50 |
| Parfum | q.s. |
| Peroxide decomposer as in examples 7 to 19 | 1.20 |
| Preservative | q.s. |
| Aqua | ad 100 |

Formulations 196 to 208—Hair wax

| | % w/w |
|---|---|
| Polyethylene glycol-6 | 30.00 |
| Polyethylene glycol-75 | 45.00 |
| Paraffin liquid | 0.50 |
| PEG-40 hydrogenated castor oil | 1.00 |
| Glycerol | 14.00 |
| Benzophenone-3 | 2.00 |
| Tocopheryl acetate | 1.00 |
| Phytantriol | 0.10 |
| Peroxide decomposer as in examples 7 to 19 | 1.00 |
| Parfum | q.s. |
| Aqua | ad 100 |

Formulations 209 to 221—Anti-dandruff hair tonic

| | % w/w |
|---|---|
| Alcohol | 45.00 |
| Aloe vera (10-fold conc.) | 1.00 |
| Panthenol | 1.00 |
| Tocopheryl acetate | 0.50 |
| PEG-40 hydrogenated castor oil | 0.50 |
| Allantoin | 0.10 |
| Hydrolyzed animal protein | 1.50 |
| 3,3-dimethyl-2-butanone | 0.30 |
| Parfum | 0.10 |
| Peroxide decomposer as in examples 7 to 19 | 1.00 |
| Aqua | ad 100 |

Formulations 222 to 234—Foot deo spray

| | % w/w |
|---|---|
| PEG-40 hydrogenated castor oil | 0.80 |
| Alcohol | 20.00 |
| Farnesol | 0.08 |
| Menthol lactate | 0.06 |
| 1,2-Propylene glycol | 3.20 |
| Benzophenone-4 | 1.20 |
| PEG-7 glyceryl cocoate | 0.80 |
| Tocopheryl acetate | 0.05 |
| Peroxide decomposer as in examples 7 to 19 | 0.01 |
| Parfum | q.s. |
| Aqua | 13.80 |
| Butane | 60.00 |

Formulations 235-247—Hair spray

| | % w/w |
|---|---|
| Aminomethyipropanol | 0.40 |
| Dimethicone copolyol | 0.03 |
| Alcohol | 43.67 |
| Pentane | 13.20 |
| Acrylates/acrylamide copolymer | 3.40 |
| Tocopheryl acetate | 1.00 |
| Peroxide decomposer as in examples 7 to 19 | 0.01 |
| Parfum | q.s. |
| Butane | 2.40 |
| Isobutane | 35.90 |

We claim:

1. A cosmetic or dermatological preparation for avoiding or reducing skin damage by peroxides or hydroperoxides formed as a result of endogenous or exogenous factors, which has a content of
a) at least one antioxidant effective as O- or C-free radical scavenger and
b) 1 to 10% by weight of an organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
where the preparation comprises, as peroxide or hydroperoxide decomposer (b), organic compounds which contain sulfur, and/or (b) aromatic amines.

2. A cosmetic or dermatological preparation as claimed in claim 1, which comprises, based on the finished preparation, 0.001 to 30% by weight of antioxidant (a).

3. A cosmetic or dermatological preparation as claimed in claim 1, which comprises, as peroxide or hydroperoxide decomposer (b), compounds which, in vitro at room temperature, dissolved in a molar concentration of 0.05 m/l in a polar or nonpolar solvent, reduce the peroxide or hydroperoxide concentration by at least 20% within 10 minutes.

4. A cosmetic or dermatological preparation as claimed in claim 1, which comprises, as peroxide or hydroperoxide decomposer (b), compounds chosen from organic sulfur compounds with a sulfur oxidation state of less than +6 and organic phosphorus compounds with a phosphorus oxidation state of less than +5.

5. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding skin damage by peroxides or hydroperoxides formed as a result of endogenous or exogenous factors.

6. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding or reducing skin damage by peroxides or hydroperoxides formed as a result of endogenous or exogenous factors as claimed in claim 5, which comprises, based on the finished preparations, 0.001 to 30% by weight of antioxidant (a) and 0.001 to 30% weight of at least one peroxide or hydroperoxide decomposer (b).

7. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding skin damage by peroxides or hydroperoxides formed as a result of endogenous or exogenous factors as claimed in claim 5, wherein sulfur- and/or phosphorus-containing compounds are used as peroxide or hydroperoxide decomposer (b).

8. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding skin damage by peroxides or hydroperoxides formed as a result of endogenous or exogenous factors as claimed in claim 5, which comprises aromatic amines as peroxide or hydroperoxide decomposer (b).

9. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding skin damage by peroxides or hydroperoxides formed as a result of endogenous or exogenous factors as claimed in claim 5, wherein the compounds (b), in vitro at room temperature, dissolved in a molar concentration of 0.05 m/l in a polar or nonpolar solvent, reduce the peroxide or hydroperoxide concentration by at least 20% within 10 minutes.

10. The use of a combination of
a) at least one antioxidant effective as O- or C-free-radical scavenger and
b) at least one organic or inorganic skin-compatible compound which reduces peroxides or hydroperoxides to the corresponding alcohols without the formation of reactive free-radical subsequent stages, where this compound is chosen such that, at body temperature, it reacts significantly more rapidly than sulfur-containing compounds intrinsic to the skin,
as additive for cosmetic or dermatological preparations for avoiding skin damage by peroxides or hydroperoxides formed as a result of endogenous or exogenous factors as claimed in claim 5, wherein the peroxide or hydroperoxide decomposer (b) used are compounds chosen from organic sulfur compounds with a sulfur oxidation state of less than +6 and organic phosphorus compounds with a phosphorus oxidation state of less than +5.

11. The use of a combination as claimed in claim 5 in cosmetic or dermatological preparations for the supplementary removal and/or alleviation of skin damage by peroxides or hydroperoxides.
